(19) United States
(12) Patent Application Publication
(10) Pub. No.: US 2019/0362819 A1
(43) Pub. Date: Nov. 28, 2019

(54) Patient data management and monitoring platform and system

(71) Applicant: Kaiku Health Oy, Helsinki (FI)

(72) Inventors: Henri VIRTANEN, Helsinki (FI); Ramin BAGHAIE, Helsinki (FI); Lauri SIPPOLA, Helsinki (FI)

(73) Assignee: Kaiku Health Oy, Helsinki (FI)

(21) Appl. No.: 15/990,686

(22) Filed: May 28, 2018

(51) Int. Cl. G16H 10/60; G16H 50/30; G16H 20/10

(52) U.S. Cl. CPC G16H 10/60; G16H 20/10; G16H 50/30

(57) ABSTRACT

Studies have consistently shown that the better patients are monitored the higher the recovery and survival rates. Due to legal & technical constraints, high quality monitoring of patients has been limited to hospitals and other care facilities. However, with the aide of the systems disclosed herein, high quality patient monitoring can be extended outside of these care facilities and thereby further increase recovery and survival rates in patients not confined to healthcare facilities. This is particularly relevant in the monitoring and treatment of chronic illnesses, for example with cancer treatment.

### FIELD OF INVENTION

The present invention relates to the field of systems and platforms for managing patient data.

### BACKGROUND OF INVENTION

Monitoring a patient's health can be directly correlated to the success of their recovery. This has typically been practiced in healthcare facilities with the use of devises and nurses which monitor patients while they are in the facility.

However, studies have now shown that with chronic illness, the more active the monitoring of long term patients, particularly outside of the healthcare facility, the higher the survivability and the longer the life expectancy.

Several problems arise in the monitoring of patients long term and outside of healthcare facilities. Some problems are legal and deal with the handling, processing and transfer of personal identifying data, which is necessary when monitoring a patient. Additionally, problems quickly arise in both the amount of data to be collected and managed as well as a single healthcare providers ability to manage data cross facilities and outside of its doors.

### SUMMARY OF THE INVENTION

It is an aspect of the invention to provide a patient data management and monitoring system.

It is an aspect of certain embodiments that the system includes an identifiable patient data processing and storage unit as well as an anonymous patient data processing and storage unit.

An identifiable patient data processing and storage unit can be capable of receiving identifiable patient data from at least a patient interface and a healthcare interface, said identifiable patient data processing unit programed to aggregate patient data from multiple independent sources including from the patient interface and the healthcare interface and store the aggregated patient data for identifiable patients.

An anonymous patient data processing and storage unit can be capable of receiving anonymous patient data, aggregating anonymous patient data and processing said aggregated anonymous patient data to be used in tailored treatment and monitoring plans for identifiable patients.

Systems can also contain an anonymizing layer which is capable of anonymizing identifiable patient data from the identifiable patient data processing and storage unit, patient interface and healthcare interface, wherein the anonymizing layer is arranged to send anonymous patient data to the anonymous patient data processing and storage unit such that anonymous patient data can be correlated to, and update existing anonymous patient data within the anonymous patient data processing and storage unit related to a same identifiable patient.

An identifiable patient data processing and storage unit can further be arranged to create and distribute tailored chronic illness monitoring plans to identifiable patients based on their aggregated identifiable patient data as well as the processed aggregated anonymous patient data, and

An identifiable patient data processing and storage unit can further be arranged to receive identifiable patient feedback from the tailored chronic illness monitoring plans, process said feedback and send processed feedback to said healthcare interface on said identifiable patients reported health status.

A system can further include a systemic therapies combination unit configured to create and distribute tailored digital treatment plans for a patient based on aggregated patient data for a patient and aggregated anonymous patient data.

Tailored digital treatment plans and tailored chronic illness monitoring plans can form a feedback loop and can be used in updating each other.

Digital treatment plans can include suggested medication and/or dosage regimes which are sent to the healthcare unit.

Digital treatment plans can also include suggesting and/or initiating a software implemented therapy for the patient.

### BRIEF DESCRIPTION OF THE DRAWINGS

FIG. 1 shows an example system for the integration and management of patient data in a healthcare environment.

FIG. 2 shows an example system for aggregating and processing patient data across multiple healthcare environments.

FIG. 3 shows an example work flow for anonymous patient data analytics.

### DETAILED DESCRIPTION OF EXEMPLARY EMBODIMENTS

Disclosed herein are patient data management and monitoring systems and examples as well as embodiments thereof. Studies have consistently shown that the better patients are monitored the higher the recovery and survival rates. Due to certain constraints, high quality monitoring of patients has been limited to hospitals and other care facilities. However, with the aide of the systems disclosed herein, high quality patient monitoring can be extended outside of these care facilities and thereby further increase recovery and survival rates in patients not confined to healthcare facilities. This is particularly relevant in the monitoring and treatment of chronic illnesses, for example with cancer treatment.

Another problem with patient data management is the privacy component. In order to best monitor patients and use that information effectively, the identity of a patient should be readily available and tied to the data. However, for legal and privacy concerns, often patient data needs to be anonymized in which case it may loose its links to other correlated data from the same patient in the past or future. Therefore, the present system handles patient data in a unique way taking into account these concerns.

A patient data management and monitoring system can include two or more different data processing and storage units. For example, one can be for, and/or contain, identifiable patient data, while the other can be for, and/or contain, anonymous patient data. Identifiable patient data here generally means that a patients personal data is tied to and/or associated with the actual patient data. Anonymous patient data here generally means that someone with access to the data would not be able to associate any particular patient data with any individual personal data.

Any data processing and storage unit of the system may be capable of receiving data from one or more sources within, or outside of the system, determining relevance of the received data, processing it in one or more predefined ways, forwarding the information either in raw or processed form to another part of the system or storing some or all of the raw or processed information. A data processing and storage unit may be one unit, it may be a portion of one unit, or it may be a separate data processing unit and storage units.

For example, an identifiable patient data processing and storage unit may be capable of receiving identifiable patient data from a patient interface and/or a healthcare interface. The identifiable patient data processing unit can be programed to aggregate patient data from multiple independent sources. These sources may include the patient interface, the healthcare interface. The unit can then store aggregated patient data for identifiable patients.

An identifiable patient data processing and storage unit may be further arranged to create and/or distribute tailored monitoring plans to identifiable patients. These tailored monitoring plans can be for chronic illnesses, such as cancer or other oncology patients. These monitoring plans can be created or chosen from a set of predefined monitoring plans based on aggregated identifiable patient data as well as processed aggregated anonymous patient data.

Monitoring plans can be based on existing heath care facility procedures and can cover monitoring of a patient both within and/or outside of a heath care facility. Additionally, monitoring plans can be unique to the present system and technology in which information can be passively or actively recorded by the patient during every day live and activities. Furthermore, monitoring plans can have a simple, linear format or the can be complex and conditional and can be chosen based off of numerous criteria from the system or system administrators. Furthermore, monitoring plans can be adaptive and be updated or changed based on received data from the system regarding the current health, location, activity or other factor related to the patient.

An identifiable patient data processing and storage unit may be further arranged to receive identifiable patient feedback from a tailored chronic illness monitoring plans. The processing unit may then process said feedback and send processed feedback forward, e.g. to a healthcare interface, healthcare database, patient interface or other predefined location with identifiable patients reported health status or possible processed warnings and/or questions.

Any patient data processing and storage unit can also be arranged to receive raw data from at least one patient sensor and optimize said raw data for storage based on a determined relevance of the sensor data to a respective patient.

One example is a sensor which is a blood pressure sensor. This may be an in-care facility sensor or a home blood pressure monitor/sensor. The sensor can send raw or processed data on a patient to an identifiable patient data processing unit. The unit may take data from the sensor to determine if the patient has an acceptable and/or expected blood pressure or if the blood pressure of the user is indicative of a health issue. If the blood pressure is within an acceptable range the data may be kept of erased. If the blood pressure is outside a predefined limit then the data can be stored and/or trigger a notification event to one or more of the system users or interfaces.

Another example is where a sensor may be a thermometer or thermocouple which sends raw temperature data on a patient or their surroundings to an identifiable patient data processing unit. The unit may take the raw data from the sensor to determine if the received temperature is over or under a predefined limit. If the received temperature is not outside the predefined limit then the sensor data can be erased. If the received temperature is outside the predefined limit then the sensor data, and/or the processed data, can be stored. The same principle applies to all other known sensors and their associated data, e.g. heart rate monitors, biometric sensors, step counters, fit bit, EKGs, geolocation and geospatial sensors, etc. In this manner, the system can self limit its own storage and information collection management.

Furthermore, an identifiable patient data processing and storage unit can be further configured to create, chose or modify a monitoring plan for a patient based on received or processed sensor data. For example, if the patient has a predictable change in blood pressure at a certain time or times during the day, then a monitoring plan can be tailored and sent to the patient to try and address the repeated issues with blood pressure. Another example couple be if a patient has normal temperature and walks a typical amount of 3-5 km per day then a first monitoring plan can be tailored and sent for the patient. When a patient has a rise in temperature, the system may also access if it is during a period of high activity in which case the system may either continue as normal in monitoring or it may increase the intervals at which the system monitors the patients temperature. If the system detects a raise in temperature and there is no, or a decreased amount of physical activity, then the system may send warnings to the patient and/or a healthcare provider or may create and/or distribute a new monitoring plan directed to the situation.

Based on the data stored in the system and the availability of both sensor data and user input data through one or more interfaces, a system can more effectively track health, such as a chronic illness. For example, if a patient has a cancer diagnosis and goes through chemotherapy multiple times, the system can track the patients' health and recovery outside of a healthcare facility before and after the therapy. Then through the system it can be determined/suggested if a different therapy may be better suited for the patient or it can be seen how a patient has reacted compared to previous cycles to determine a long term predictive patient assessment.

An identifiable patient data processing and storage unit may further be configured to send raw, processed or optimized sensor data to the healthcare interface based on a determined relevance, as discussed above. The system may also push any other raw or processed data, monitoring plan or other information to a healthcare interface or database. In particular though, the system can help manage a healthcare providers time by only pushing determined relevant information so that they are not inundated with non-relevant information, which may otherwise be maintained in the system storage.

Relevance of sensor data can be determined at least in part based on a patient's received feedback from a monitoring plan and/or by a request from a healthcare interface for specific data on a patient.

To continue the first example, a rise in blood pressure may simply be related to the time of measuring in relation to meals. Therefore, a questionnaire can be pushed to the user interface which may be as simple as, “An unexpected change in blood pressure has been recorded. Have you eaten a meal in the last 30 minutes.” The questionnaire may be more in depth and ask about meal contents such as if it is high in salt or if the patient has experiences an abnormal amount of stress recently.

To continue the other example, a rise in temperature of a patient on its own can be relevant without any other data. However, if there is no geolocation sensor also associated with a patient, a questionnaire can be pushed to the user interface. This questionnaire may ask about the patients recent physical activity. From this information, a relevance of the temperature data may be readily ascertainable, for example if they patient has just come back from a run in the heat outside. However, by combining all of the information available to the system, the system may determine that even though there has been recent activity, the temperature rose or otherwise acted in a different manner than other times with similar activity, or the temperature rise happened at an expected time with regards to a therapy or other event, and thus may still be relevant for a healthcare provider.

An anonymous patient data processing and storage unit can be capable of some or all of: receiving anonymous patient data, aggregating anonymous patient data and processing said aggregated anonymous patient data to be used in tailored treatment and monitoring plans for identifiable patients.

The system may also contain a systemic therapies combination unit. Such a unit can be configured to create, modify, select and/or distribute tailored digital treatment plans for a patient. These tailored digital treatment plans can be based on aggregated patient data for a patient and/or aggregated anonymous patient data. Additionally, a tailored digital treatment plans and a monitoring plans can form a feedback loop and thereby can be used in updating one or both of the other.

Systemic therapies can be physical therapies of patients. Therapy information can be one source of information which helps a processing unit to activate a proper treatment monitoring plan or follow-up module. Digital treatment plans may also include suggested medication and/or dosage regimes. Digital treatment plans may also include suggesting and/or initiating a software implemented therapy for the patient. Digital treatment plans may include at least one suggested physical activity for a patient. These digital treatment plans can be sent to a healthcare unit, one or more of the system interfaces, or one or more of the system databases & storage units.

Digital treatment plans may also include at least one suggested physical activity for a patient. When a patient has a relevant sensor available, then sensor data can be used to track implementation of all or a portion of the digital treatment plan.

Feedback from a digital treatment plan or a monitoring plan can be processed by the identifiable patient data processing and storage unit to include a determination of a relevance value for at least some of the feedback. Based on the relevance value data, raw or processed can be selectively stored. For example, only a portion of feedback maybe stored in the identifiable patient data storage unit and/or the anonymous patient data storage unit. The stored portion of the feedback can be stored in such a way that an inference about non-stored feedback can be made from the stored portion of the feedback.

A system can also contain an anonymizing layer. An anonymizing layer can anonymizes identifiable patient data, for example from an identifiable patient data processing and storage unit, patient interface, healthcare interface or other database. An anonymizing layer can be arranged to send anonymous patient data to an anonymous patient data processing and storage unit. Furthermore, the anonymizing layer can send the data in such a manner that anonymous patient data can be correlated to, and/or update existing anonymous patient data within the anonymous patient data processing and storage unit related to a same identifiable patient.

An anonymizing layer can be configured such that when an identifiable patent's data is entered, and wherein there is existing anonymized patient data which originally related to said patient, the anonymizing layer is capable of updating the related anonymized patient data with the identifiable patient's new data in an anonymized format. This can be done through an appropriate anonymizing algorithm and data structure.

A healthcare interface or another portion of the system, e.g. the identifiable patient data processing and storage unit may include directly and/or access to, at least one secure healthcare database. The system may further include an application program interface capable of pulling identifiable patient data for identifiable patients with data already stored in the identifiable patient data storage unit. This may be from a healthcare database containing information on patients both within or not within the identifiable patient data storage unit.

A healthcare interface or another portion of the system, e.g. the identifiable patient data processing and storage unit may include directly and/or access to, at least one secure healthcare database. The patient data management system may further include an application program interface capable of pulling anonymous patient data to the anonymous patient data processing and storage unit.

An anonymous patient data processing and storage unit can have access to anonymized data from at least one clinical data database. Processing aggregated anonymous patient data to be used in tailored treatment and monitoring plans can include determining a relevance of anonymous data pulled from the clinical data database and incorporating relevant anonymous data to the processed aggregated anonymous patient data.

The system can also include a clinical data processing and storage unit configured to create mock clinical trials. It can do so by querying stored data from the anonymous patient data storage unit and supplementing said data by causing the identifiable patient data processing and storage unit to create tailored monitoring plans designed to generate desired supplemental data.

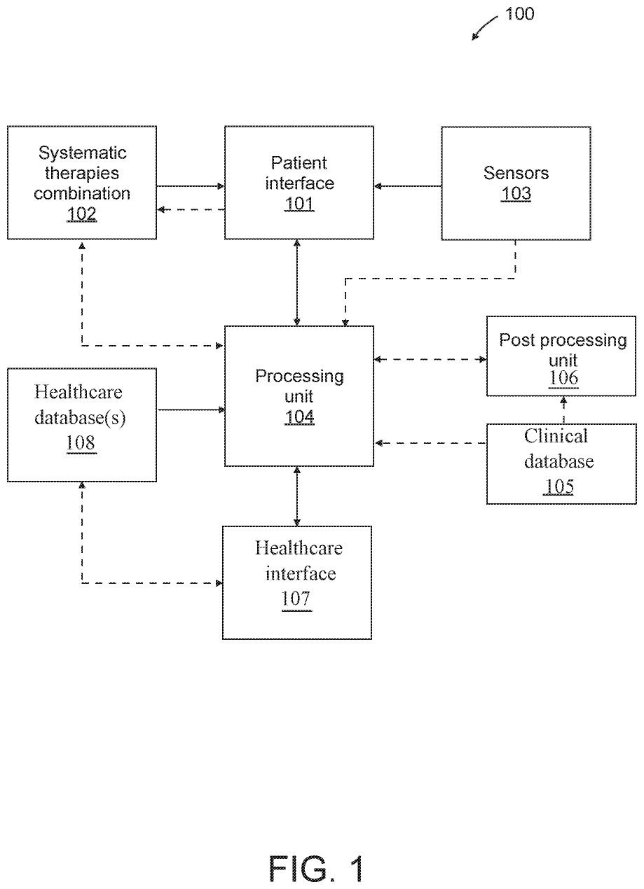

FIG. 1 shows an example system 100 for the integration and management of patient data in a healthcare environment. A patient interface 101 and/or a healthcare interface 107 can be any electronic device in which a patient or healthcare provider respectively can interact with the platform including mobile phones, tablets, computers, browser based products, etc. These can be portions of the system both within a healthcare facility and/or outside of a healthcare facility.

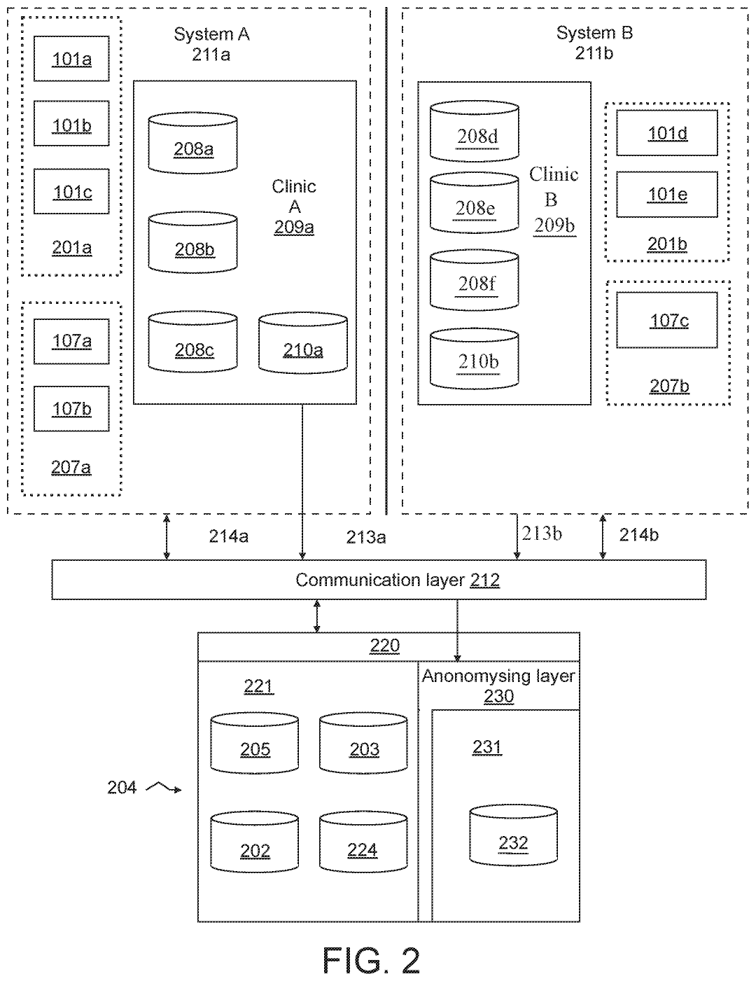

The patient interface 101 and the healthcare interface 107 are the two main user interfaces of the system and can communicate directly with each other, or they can communicate through each other through a processing unit 104. The processing unit 104 can be, or can contain, one or more of the patient processing and storage units discussed above. The processing unit 104 can be a computer, server or set of computers and servers, local or distributed. The processing unit 104 can be a single system which incorporates and/or stores data from all other parts of the system or it can be a group of connected systems. This is also discussed in more detail in portion 204 of FIG. 2.

Also in communication with the processing unit 104, either directly or indirectly, are the other portions system including any sensors 103, a systemic therapies combination unit 102, a post processing unit 106, and any databases such as healthcare databases 108 and clinical data databases 105.

Generally, systemic therapies combinations from the unit 102 can be therapy plans, computer programs, digital therapies, pipelines, etc. These can be stand alone computer implemented therapies or they can be paired with medication and/or standard physical therapies.

Sensors 103 can include all known sensors including biometric sensors, medical sensors and personal sensors. For example, sensors in place in healthcare environments currently can be integrated in to the present platforms or some or all of their data can be accessible to or imported to the present platforms.

Clinical data database 105 can be a container or database of clinical data. The data contained herein may be anonymized or contain personal data. While it may contain data on patients who are a part of the system, e.g. who have access to a patient interface 101, it may contain information on others who are do not have access to the system 101, and it can be data from a specific study or studies which can be used for analysis purposes. The data may also be from Real World Data (RWD).

The post processing unit 106 can be a component of the platform which takes information from the rest of the system and can create predictive and/or suggestive care decisions for system users. For example, monitoring plans can be created in either the processing unit 104 or the post processing unit 106. Portions of the system can be modular and moved around as desired. For example, the processing unit 104 may contain all, or most of the identifiable patient data processing and storage unit while the post processing unit 106 may contain most or all of the anonymized patient data processing and storage unit. Suggested interactions and communications between the units can be seen in the figure, however the communication of the units is not limited thereto.

The present system, or examples thereof, can be healthcare facility independent. As such, the system can include patients, doctors and information from multiple facilities and compile it for use in a single system, as shown in FIG. 2.

FIG. 2 shows two healthcare systems, system A 211a and system B 211b. Each healthcare system can contain a set of patient interfaces 201a and 201b respectively which contain individual patient interfaces 101a-e. In some cases, a patient interface can be in more than on set of patient interfaces, for example if a patient visits multiple healthcare facilities. Similarly, each system includes a plurality of healthcare interfaces 107a-c in sets 207a and 2017b. The patient and healthcare interfaces may be limited to the particular facility or system or they may be able to move around multiple areas and systems.

Each system contains data from the clinic 209a and 209b respectively. This data can be stored in a variety of separate or linked databases 208a-f and 210a-b. Examples of these databases are OIS, EHR and LIS but are not limited thereto.

In general, different clinics information tend to be separate from each other. Therefore, the present system can include a communications layer 212 which can communicate either directly 213a and 213b or indirectly 214a and 214b with each portion of the associated systems. While FIG. 2 is shown with two systems A and B, there may be a plurality of additional systems.

The communication layer 212 can communicate directly with a processing unit 220 or with a anonymizing layer 230. The anonymizing layer 230 is used as discussed above to remove personal identifying data from patient data and optionally to keep the anonymized data in a format which can be updated based on future patient data from the same source and/or individual. The system 204 can contain an identifiable processing unit 221 and an anonymous processing unit 231. The system 204 can contain multiple units and/or databases such as a monitoring plan database 205, an identifiable patient data storage unit 203, a systems therapies unit 202, and a mock trial unit 224. On the anonymous side there can also be one or more storage units or databases 232.

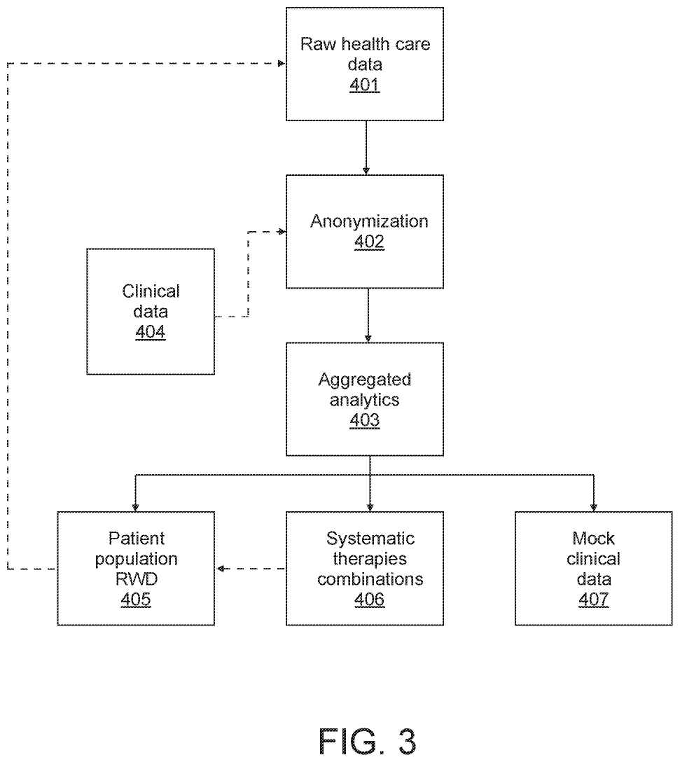

In order to maximize utility of the system, the identifiable and anonymous aggregated information should work together. Therefore, it is best when anonymized aggregated data is smart data and is able to track progress of the unidentifiable data points through time. FIG. 3 shows how raw healthcare data 401 from any portion of the system as described herein can be sent to an anonymization layer, protocol or algorithm 402. Additionally, clinical data 404 can be also sent to anonymization 402. 402 can simply strip personal and/or identifiable information from patient data or it may include an advanced process or algorithm which, while removing the identifiability of the data still maintains a way for future data related to that individual to be tied to the corresponding anonymized information. In this way, the aggregated anonymous data can be updated as patient data is gathered.

Once the healthcare data is anonymized then it is possible to perform aggregated analytics on the anonymized data 403. One benefit to this is that it is possible to analyze anonymized data in different ways, with different methods and at different locations than it is with data which contains identifiable personal data due to legal restrictions. From here, information or aggregated anonymous data can be sent to, or used by different sources including, but not limited to, patient population Real World Data (RWD) database, systemic therapies combination unit 406, mock clinical data database or unit 407 or back to the identifiable side 221 of the system 204 to be used on further tailoring monitoring plans for users.

Furthermore, there can be a non-transitory computer readable medium having stored thereon a set of computer readable instructions for causing a processor of a computing device to carry out the methods and steps described above.

It is to be understood that the embodiments of the invention disclosed are not limited to the particular structures, process steps, or materials disclosed herein, but are extended to equivalents thereof as would be recognized by those ordinarily skilled in the relevant arts. It should also be understood that terminology employed herein is used for the purpose of describing particular embodiments only and is not intended to be limiting.

Reference throughout this specification to “one embodiment” or “an embodiment” means that a particular feature, structure, or characteristic described in connection with the embodiment is included in at least one embodiment of the present invention. Thus, appearances of the phrases “in one embodiment” or “in an embodiment” in various places throughout this specification are not necessarily all referring to the same embodiment.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary. In addition, various embodiments and example of the present invention may be referred to herein along with alternatives for the various components thereof. It is understood that such embodiments, examples, and alternatives are not to be construed as de facto equivalents of one another, but are to be considered as separate and autonomous representations of the present invention.

Furthermore, the described features, structures, or characteristics may be combined in any suitable manner in one or more embodiments. In the following description, numerous specific details are provided, such as examples of lengths, widths, shapes, etc., to provide a thorough understanding of embodiments of the invention. One skilled in the relevant art will recognize, however, that the invention can be practiced without one or more of the specific details, or with other methods, components, materials, etc. In other instances, well-known structures, materials, or operations are not shown or described in detail to avoid obscuring aspects of the invention.

While the forgoing examples are illustrative of the principles of the present invention in one or more particular applications, it will be apparent to those of ordinary skill in the art that numerous modifications in form, usage and details of implementation can be made without the exercise of inventive faculty, and without departing from the principles and concepts of the invention. Accordingly, it is not intended that the invention be limited, except as by the claims set forth below.

1. A patient data management system comprising;
an identifiable patient data processing and storage unit capable of receiving identifiable patient data from at least a patient interface and a healthcare interface, said identifiable patient data processing unit programed to aggregate patient data of multiple patients from multiple independent sources including from the patient interface and the healthcare interface and store the aggregated patient data for identifiable patients,
an anonymous patient data processing and storage unit capable of receiving anonymous patient data, aggregating anonymous patient data of multiple patients and processing said aggregated anonymous patient data to be used in tailored treatment and monitoring plans for identifiable patients,
an anonymizing layer which anonymizes identifiable patient data from the identifiable patient data processing and storage unit, patient interface and healthcare interface, wherein the anonymizing layer is arranged to send anonymous patient data to the anonymous patient data processing and storage unit such that anonymous patient data can be correlated to, and update existing anonymous patient data within the anonymous patient data processing and storage unit related to a same identifiable patient, and
a systemic therapies combination unit configured to create and distribute tailored digital treatment plans for a patient based on aggregated patient data for a patient and aggregated anonymous patient data, wherein the identifiable patient data processing and storage unit is further arranged to create and distribute tailored chronic illness monitoring plans to identifiable patients based on their aggregated identifiable patient data as well as the processed aggregated anonymous patient data, and
wherein the identifiable patient data processing and storage unit is further arranged to receive identifiable patient feedback from the tailored chronic illness monitoring plans, process said feedback and send processed feedback to said healthcare interface on said identifiable patients reported health status,
wherein processing of feedback by the identifiable patient data processing and storage unit includes determination of a relevance value for at least some of the feedback and selectively storing data based on it's determined relevance value,
wherein the tailored digital treatment plans and tailored chronic illness monitoring plans form a feedback loop and are used in updating each other, and
wherein digital treatment plans include suggesting and/or initiating a software implemented therapy for the patient.

2. The patient data management system of claim 1, further comprising;
wherein the identifiable patient data processing and storage unit and/or the anonymous patient data processing and storage unit are arranged to receive raw data from at least one patient sensor and optimize said raw data for storage based on a determined relevance of the sensor data to a respective patient, and
wherein the identifiable patient data processing and storage unit is further configured to create the tailored chronic illness monitoring plan for a patient based on the sensor data, and
wherein the identifiable patient data processing and storage unit is further configured to send optimized sensor data to the healthcare interface based on its determined relevance.

3. The patient data management system of claim 2, wherein relevance of sensor data is determined at least in part based on a patient's received feedback from a tailored chronic illness monitoring plan and/or by a request from a healthcare interface for specific data on a patient.

4. (canceled)

5. The patient data management system of claim 1, wherein digital treatment plans include suggested medication and/or dosage regimes which are sent to the healthcare unit.

6. (canceled)

7. The patient data management system of claim 1, wherein digital treatment plans include at least one suggested physical activity for a patient.

8. The patient data management system of claim 2, wherein the digital treatment plans include at least one suggested physical activity for a patient, and wherein patient sensor data is used to track implementation of a portion of the digital treatment plan.

9. The patient data management system of claim 1, wherein the anonymizing layer is configured such that when an identifiable patient's data is entered, and wherein there is existing anonymized patient data which originally related to said patient, the anonymizing layer is capable of updating the related anonymized patient data with the identifiable patient's new data in an anonymized format.

10. (canceled)

11. The patient data management system of claim 1, wherein only a portion of the feedback is stored in the identifiable patient data storage unit and/or the anonymous patient data storage unit, and said portion of the feedback is stored in such a way that an inference about non-stored feedback can be made from the stored portion of the feedback.

12. The patient data management system of claim 1, wherein the healthcare interface includes directly and/or access to, at least one secure healthcare database, and wherein the patient data management system further comprises an application program interface capable of pulling identifiable patient data for identifiable patients with data already stored in the identifiable patient data storage unit.

13. The patient data management system of claim 1, wherein the healthcare interface includes directly and/or access to, at least one secure healthcare database, and wherein the patient data management system further comprises an application program interface capable of pulling anonymous patient data to the anonymous patient data processing and storage unit.

14. The patient data management system of claim 1, wherein the anonymous patient data processing and storage unit has access to anonymized data from at least one clinical data database, and wherein processing aggregated anonymous patient data to be used in tailored treatment and monitoring plans includes determining a relevance of anonymous data pulled from the clinical data database and incorporating relevant anonymous data to the processed aggregated anonymous patient data.

15. The patient data management system of claim 1, further comprising a clinical data processing and storage unit configured to create mock clinical trials by querying stored data from the anonymous patient data storage unit and supplementing said data by causing the identifiable patient data processing and storage unit to create tailored chronic illness monitoring plans designed to generate desired supplemental data.
